(19) United States
(12) Patent Application Publication
(10) Pub. No.: US 2007/0014751 A1
(43) Pub. Date: Jan. 18, 2007

(54) Novel spiro-quinuclidinyl derivatives for the treatment of central nervous system disorders

(76) Inventors: Yifang Huang, Lansdale, PA (US); Michael H. Parker, Chalfont, PA (US); Allen B. Reitz, Lansdale, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/443,929

(22) Filed: May 31, 2006

Related U.S. Application Data
(60) Provisional application No. 60/695,316, filed on Jun. 30, 2005.

(51) Int. Cl. A61K 31/74

(52) U.S. Cl. 424/78.08

(57) ABSTRACT

The present invention is directed to novel spiro-quinuclidinyl derivatives, pharmaceutical compositions containing them and their use in the treatment of central nervous system disorders.

### CROSS REFERENCE TO RELATED APPLICATIONS

This application claims the benefit of U.S. Provisional Application 60/695,316, filed on Jun. 30, 2005, which is incorporated by reference herein in its entirety.

### FIELD OF THE INVENTION

The present invention is directed to novel spiro-quinuclidinyl derivatives, pharmaceutical compositions containing them and their use in the treatment of central nervous system disorders. The compounds of the invention are useful for the treatment of central nervous system disorders, including Alzheimer's disease (AD), mild cognitive impairment, senility, dementia, dementia with Lewy bodies, Down's syndrome, dementia associated with Parkinson's disease and dementia associated with beta-amyloid.

### BACKGROUND OF THE INVENTION

Alzheimer's Disease (AD) is a neurodegenerative disease associated with aging. AD patients suffer from cognition deficits and memory loss as well as behavioral problems such as anxiety. Over 90% of those afflicted with AD have a sporadic form of the disorder while less than 10% of the cases are familial or hereditary. In the United States, about 1 in 10 people at age 65 have AD while at age 85, 1 out of every two individuals are affected with AD. The average life expectancy from the initial diagnosis is 7-10 years, and AD patients require extensive care either in an assisted living facility which is very costly or by family members. With the increasing number of elderly in the population, AD is a growing medical concern. Currently available therapies for AD merely treat the symptoms of the disease and include acetylcholinesterase inhibitors to improve cognitive properties as well as anxiolytics and antipsychotics to control the behavioral problems associated with this ailment.

The hallmark pathological features in the brain of AD patients are neurofibillary tangles which are generated by hyperphosphorylation of tau protein and amyloid plaques which form by aggregation of β-amyloid₁₋₄₂ (Aβ₁₋₄₂) peptide. Aβ₁₋₄₂ forms oligomers and then fibrils, and ultimately amyloid plaques. The fibrils are believed to be especially neurotoxic and may cause most of the neurological damage associated with AD. Thus, agents that prevent the formation of Aβ₁₋₄₂ fibrils, aggregates and/or plaques have the potential to be disease-modifying agents for the treatment of AD, mild cognitive impairment and dementia.

### SUMMARY OF THE INVENTION

The present invention is directed to a compound of formula (I)

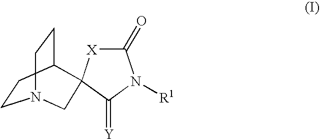

wherein

X is selected from the group consisting of O and NR^{A}; and Y is selected from the group consisting of O and NR^{B}; wherein R^{A} and R^{B} are each selected from the group consisting of hydrogen and lower alkyl;

provided that when X is O, then Y is O;

provided that when X is NR^{A} and Y is NR^{B} then R^{A} and R^{B} are each hydrogen;

R¹ is selected from the group consisting of alkyl, alkoxycarbonyl and aryl; wherein the aryl is optionally substituted with one or more substituents independently selected from halogen, hydroxy, carboxy, alkyl, alkoxy, nitro, cyano, NR^{C}R^{D}, halogenated lower alkyl, halogenated lower alkoxy, alkoxycarbonyl or —S(O)₀₋₂-alkyl;

wherein each R^{C} and R^{D} is independently selected from hydrogen or lower alkyl;

provided that when X is NH and Y is O, then R¹ is other than methyl;

or a pharmaceutically acceptable salt thereof.

The present invention is further directed to a compound of formula (II)

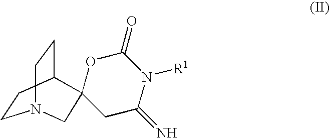

wherein

R¹ is selected from the group consisting of alkyl, alkoxycarbonyl and aryl; wherein the aryl is optionally substituted with one or more substituents independently selected from halogen, hydroxy, carboxy, alkyl, alkoxy, nitro, cyano, NR^{C}R^{D}, halogenated lower alkyl, halogenated lower alkoxy, alkoxycarbonyl or —S(O)₀₋₂-alkyl;

wherein each R^{C} and R^{D} is independently selected from hydrogen or lower alkyl;

or a pharmaceutically acceptable salt thereof.

Illustrative of the invention is a pharmaceutical composition comprising a pharmaceutically acceptable carrier and any of the compounds described above. An illustration of the invention is a pharmaceutical composition made by mixing any of the compounds described above and a pharmaceutically acceptable carrier. Illustrating the invention is a process for making a pharmaceutical composition comprising mixing any of the compounds described above and a pharmaceutically acceptable carrier.

Exemplifying the invention are methods of treating central nervous system disorders in a subject in need thereof comprising administering to the subject a therapeutically effective amount of any of the compounds or pharmaceutical compositions described above.

An example of the invention is a method for a central nervous system disorder selected from the group consisting of Alzheimer's disease (AD), mild cognitive impairment, senility, dementia, dementia with Lewy bodies, Down's syndrome, dementia associated with Parkinson's disease and dementia associated with beta-amyloid, in a subject in need thereof comprising administering to the subject an effective amount of any of the compounds or pharmaceutical compositions described above.

Another example of the invention is the use of any of the compounds described above in the preparation of a medicament for treating: (a) Alzheimer's Disease (AD), (b) mild cognitive impairment, (c) senility, (d) dementia, (e) dementia with Lewy bodies, (f) Down's syndrome, (g) dementia associated with Parkinson's disease and (h) dementia associated with beta-amyloid, in a subject in need thereof.

### DETAILED DESCRIPTION OF THE INVENTION

The present invention is directed to novel spiro-quinuclidinyl derivatives, compounds of formula (I) and compounds of formula (II)

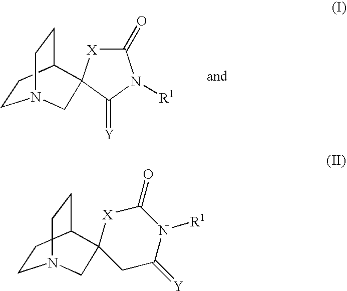

wherein X, Y, R⁰ and R¹ are as herein defined. The compounds of the present invention are useful for the treatment of central nervous system disorders, including but not limited to, Alzheimer's disease (AD), mild cognitive impairment, senility, dementia, dementia with Lewy bodies, Down's syndrome, dementia associated with Parkinson's disease and dementia associated with beta-amyloid. Preferably, the coimpounds of the present invention are useful in the treatment of Alzheimer's disease (AD), mild cognitive impairment or demetion, more preferably, Alzheimer's disease (AD) or mild cognitive impairment.

In an embodiment of the present invention, X is O and Y is O. In another embodiment of the present invention X is NH and Y is O. In yet another embodiment of the present invention X is NH and Y is NH.

In an embodiment of the present invention, R^{A} and R^{B} are each independently selected from hydrogen or methyl. Preferably R^{A} and R^{B} are each hydrogen.

In an embodiment of the present invention, R¹ is selected from the group consisting of lower alkyl, lower alkoxycarbonyl-(lower alkyl), aryl and substituted aryl; wherein the substituents on the aryl group are one to two independently selected from hydroxy, halogen, lower alkyl, lower alkoxy, trifluoromethyl, nitro, cyano, lower alkoxycarbonyl, lower alkylthio or lower alkylsulfonyl. Preferably, R¹ is selected from the group consisting of ethyl, ethoxycarbonylmethyl, phenyl, 3-hydroxyphenyl, 4-hydroxyphenyl, 2-fluorophenyl, 3-fluorophenyl, 2-chlorophenyl, 3-chlorophenyl, 2-methylphenyl, 4-methylphenyl, 2-isopropylphenyl, 2-methoxyphenyl, 3-methoxyphenyl, 4-methoxyphenyl, 2-trifluoromethylphenyl, 3-trifluoromethylphenyl, 4-trifluoromethylphenyl, 3-nitrophenyl, 3-cyanophenyl, 4-cyanophenyl, 3-ethoxycarbonylphenyl, 4-hydroxy-2-methylphenyl, 3-chloro-4-methylphenyl, 2-methyl-4-methoxyphenyl , 4-methoxy-2-methylphenyl, 2,3-dimethylphenyl, 2,5-dimethylphenyl, 3,4-dimethylphenyl, 3,5-dimethylphenyl, 2,4-dimethoxphenyl, 2,5-dimethoxyphenyl, 3,5-dimethoxyphenyl, 2,4-dihydroxyphenyl, 3,4-dichlorophenyl, 3,5-dichlorophenyl, 2,5-dichlorophenyl, 2,4-difluorophenyl, 3,5-bis(trifluoromethyl)phenyl, 3,5-di(methoxycarbonyl)phenyl, 4-methylthiophenyl, 4-methylsulfonylphenyl and 1-naphthyl.

In an embodiment of the present invention, R¹ is selected from the group consisting of lower alkyl, lower alkoxycarbonyl-(lower alkyl), aryl and substituted aryl; wherein the substituents on the aryl are one to two substituents independently selected from hydroxy, halogen, lower alkyl, lower alkoxy, trifluoromethyl, nitro, cyano, lower alkoxycarbonyl, lower alkylthio or lower alkylsulfonyl. Preferably, R¹ is selected from the group consisting of ethyl, ethoxycarbonylmethyl, 3-hydroxyphenyl, 4-hydroxyphenyl, 2-fluorophenyl, 2-chlorophenyl, 3-chlorophenyl, 3-fluorophenyl, 2-methylphenyl, 4-methylphenyl, 2-isopropylphenyl, 3-methoxyphenyl, 4-methoxyphenyl, 2-trifluoromethylphenyl, 3-trifluoromethylphenyl, 4-trifluoromethylphenyl, 3-nitrophenyl, 3-cyanophenyl, 4-cyanophenyl, 3-ethoxycarbonylphenyl, 4-hydroxy-2-methylphenyl, 3-chloro-4-methylphenyl, 4-methoxy-2-methylphenyl, 2,3-dimethyiphenyl, 2,5-dimethylphenyl, 3,4-dimethylphenyl, 3,5-dimethylphenyl, 2,4-dimethoxphenyl, 2,5-dimethoxyphenyl, 2,4-dihydroxyphenyl, 3,4-dichlorophenyl, 2,5-dichlorophenyl, 2,4-difluorophenyl, 3,5-bis(trifluoromethyl)phenyl, 3,5-di(methoxycarbonyl)phenyl, 4-methylthiophenyl, 4-methylsulfonylphenyl and 1-naphthyl.

In an embodiment of the present invention, R¹ is selected from the group consisting of aryl and substituted aryl; wherein the substituents on the aryl are one to two independently selected from halogen, trifluoromethyl, lower alkyl, lower alkoxy or lower alkoxycarbonyl. Preferably R¹ is selected from the group consisting of 2-fluorophenyl, 3-trifluoromethylphenyl, 4-trifluoromethylphenyl, 3-ethoxycarbonylphenyl, 3,4-dichlorophenyl, 3,4-dimethylphenyl, 2,4-dimethoxyphenyl, 3,5-bis(trifluoromethyl)phenyl and 1-naphthyl.

In an embodiment of the present invention, R¹ is selected from the group consisting of aryl and substituted aryl; wherein the substituents on the aryl are one to two independently selected from halogen, lower alkyl, lower alkoxy, trifluoromethyl or lower alkoxycarbonyl. Preferably, R¹ is selected from the group consisting of phenyl, 3-chlorophenyl, 4-methylphenyl, 2-methoxyphenyl, 3-methoxyphenyl, 4-methoxyphenyl, 3-trifluoromethylphenyl, 3-ethoxycarbonylphenyl, 2,4-difluorophenyl, 3,4-dichlorophenyl, 3,5-dichlorophenyl, 3,5-dimethylphenyl, 2,4-dimethoxyphenyl, 3,5-dimethoxyphenyl, 3-chloro-4-methylphenyl and 2-methyl-4-methoxyphenyl.

In an embodiment of the present invention R¹ is other than methyl. In another embodiment of the present invention, when X is NH and Y is O, then R¹ is other than lower alkyl.

Representative compounds of the present invention were prepared as listed in Tables 1-3.

TABLE 1

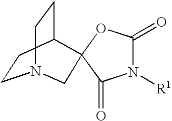

| ID No. | R¹ |
|---|---|
| 1 | 3-trifluoromethylphenyl |
| 2 | 4-methylphenyl |
| 3 | 4-methoxyphenyl |
| 4 | 4-methoxy-2-methylphenyl |
| 5 | 3,4-dichlorophenyl |
| 6 | 3-nitrophenyl |
| 7 | 3,5-dichlorophenyl |
| 8 | 3,5-bis(trifluoromethyl)phenyl |
| 9 | 2-fluorophenyl |
| 10 | 3-chlorophenyl |
| 11 | 2,5-dimethylphenyl |
| 12 | 2,5-dichlorophenyl |
| 13 | 3-ethoxycarbonylphenyl |
| 14 | 3-methoxyphenyl |
| 15 | 3-hydroxyphenyl |
| 16 | 3,4-dimethylphenyl |
| 17 | 2,4-dihydroxyphenyl |
| 18 | 4-hydroxyphenyl |
| 19 | 4-hydroxy-2-methylphenyl |
| 20 | 3,5-dimethylphenyl |
| 21 | 2,4-difluorophenyl |
| 22 | 2-chlorophenyl |
| 23 | 2,4-dimethoxyphenyl |
| 24 | 3-chloro-4-methylphenyl |
| 25 | 3-fluorophenyl |
| 26 | 2-methylphenyl |
| 27 | 2,3-dimethylphenyl |
| 28 | 2-isopropylphenyl |
| 29 | 2,5-dimethoxyphenyl |
| 30 | ethoxycarbonylmethyl |
| 31 | 1-naphthyl |
| 32 | 4-cyanophenyl |
| 33 | 4-trifluoromethylphenyl |
| 34 | 2-trifluoromethylphenyl |
| 35 | 4-methylthiophenyl |
| 36 | 4-methylsulfonylphenyl |
| 37 | ethyl |
| 38 | 3,5-di(methoxycarbonyl)phenyl |
| 39 | 3-cyanophenyl |

TABLE 2

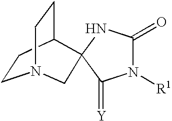

| ID No. | R¹ | Y |
|---|---|---|
| 40 | 3-trifluoromethylphenyl | O |
| 41 | 1-naphthyl | O |
| 42 | 4-trifluoromethylphenyl | O |
| 43 | 3,5-bis-(methoxycarbonyl)phenyl | O |
| 44 | 3,5-bis(trifluoromethyl)-phenyl | O |
| 45 | 2-fluorophenyl | O |
| 46 | 3-ethoxycarbonylphenyl | O |
| 47 | 3,4-dimethylphenyl | O |
| 48 | 3,4-dichlorophenyl | O |
| 49 | 2,4-dimethoxyphenyl | NH |

TABLE 3

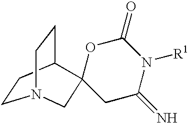

| ID No. | R¹ |
|---|---|
| 100 | 4-methoxyphenyl |
| 101 | 4-methylphenyl |
| 102 | 3-methoxyphenyl |
| 103 | 3-trifluoromethylphenyl |
| 104 | 3,4-dichlorophenyl |
| 105 | 2,4-dimethoxyphenyl |
| 106 | 3,5-dimethylphenyl |
| 107 | 3,5-dichlorophenyl |
| 108 | 3-chlorophenyl |
| 109 | 3-chloro-4-methylphenyl |
| 110 | 3-ethoxycarbonylphenyl |
| 111 | 3,5-dimethoxyphenyl |
| 112 | phenyl |
| 113 | 2-methyl-4-methoxyphenyl |
| 114 | 2-methoxyphenyl |
| 115 | 2,4-difluorophenyl |

As used herein, “halogen” shall mean chlorine, bromine, fluorine and iodine.

As used herein, the term “alkyl” whether used alone or as part of a substituent group, include straight and branched chains. For example, alkyl radicals include methyl, ethyl, propyl, isopropyl, butyl, isobutyl, sec-butyl, t-butyl, pentyl and the like. Unless otherwise noted, “lower” when used with alkyl means a carbon chain composition of 1-4 carbon atoms.

As used herein, unless otherwise noted, “alkoxy” shall denote an oxygen ether radical of the above described straight or branched chain alkyl groups. For example, methoxy, ethoxy, n-propoxy, sec-butoxy, t-butoxy, n-hexyloxy and the like. Unless otherwise noted, “lower” when used with alkoxy means an oxygen ether radical 1-4 carbon atoms.

As used herein, unless otherwise noted, the term “halogenated lower alkyl” shall mean any.lower alkyl group as defined above substituted with at least one halogen atom, preferably substituted with a least one fluoro atom. Suitable examples include but are not limited to —CF₃, —CH₂-CF₃, —CF₂-CF₂—CF₂-CF₃, and the like.

Similarly, unless otherwise noted, the term “halogenated lower alkoxy alkyl” shall mean any lowerr alkoxy group as defined above substituted with at least one halogen atom, preferably substituted with a least one fluoro atom. Suitable examples include but are not limited to —OCF₃, —OCH₂-CF₃, —OCF₂—CF₂-CF₂—CF₃, and the like.

As used herein, unless otherwise noted, “aryl” shall refer to unsubstituted carbocyclic aromatic groups such as phenyl, naphthyl, and the like, preferably phenyl.

When a particular group is “substituted” (e.g., Phe, aryl, heteroalkyl, heteroaryl), that group may have one or more substituents, preferably from one to five substituents, more preferably from one to three substituents, most preferably from one to two substituents, independently selected from the list of substituents.

With reference to substituents, the term “independently” means that when more than one of such substituents is possible, such substituents may be the same or different from each other.

Under standard nomenclature used throughout this disclosure, the terminal portion of the designated side chain is described first, followed by the adjacent functionality toward the point of attachment. Thus, for example, a “phenyl-C₁-C₆alkyl-aminocarbonyl-C₁-C₆alkyl” substituent refers to a group of the formula

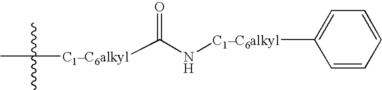

Abbreviations used in the specification, particularly the Schemes and Examples, are as follows:
AcH=Acetylcholinesterase
DMF=N,N-Dimethylformamide
HPLC=High Pressure Liquid Chromatography
MW=Molecular Weight
THF=Tetrahydrofuran

The term “subject” as used herein, refers to an animal, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or -potassium salts; alkaline earth metal salts, e.g., calcium or magnesium salts; and salts formed with suitable organic ligands, e.g., quaternary ammonium salts. Thus, representative pharmaceutically acceptable salts include the following:

acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methyinitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids and bases which may be used in the preparation of pharmaceutically acceptable salts include the following:

acids including acetic acid, 2,2-dichloroactic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydrocy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hipuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid,1-hydroxy-2-naphthoic acid, nicotinc acid, nitric acid, oleic acid, orotic acid, oxalic acid, palmitric acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid; and

bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in Design of Prodrugs, ed. H. Bundgaard, Elsevier, 1985.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistrv, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

Compounds of formula (I) wherein X is 0 and Y is 0 may be prepared according to the process outlined in Scheme 1.

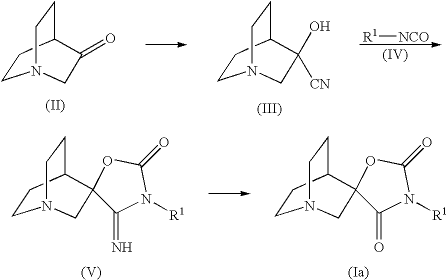

Accordingly, a compound of formula (II) as its corresponding HCI salt, a known compound or compound prepared by known methods, is reacted with a source of cyanide such as potassium cyanide, sodium cyanide, and the like, in water, at about 0° C., to yield the corresponding compound of formula (III).

Alternatively, a compound of formula (II), a known compound or compound prepared by known methods, is reacted with TMSCN, in an organic solvent such as dichloromethane, chloroform, dichloroethane, and the like, at about room temperature, to yield the corresponding compound of formula (III).

The compound of formula (III) is reacted with a suitably substituted isocyanate derivative, a compound of formula (III), a known compound or compound prepared by known methods, preferably in the presence of a catalyst such as CuCl, CuBr, CuI, in an organic solvent such as DMF, DMSO, and the like, preferably in a polar organic solvent which at least partially dissolves the catalyst, at a temperature greater than room temperature, preferably at a temperature in the range of about 60° C. to about 120° C., more preferably at about 100° C., to yield the corresponding compound of formula (V).

The compound of formula (V) is reacted with an acid such as concentrated HCl, sulfonic acid, sulfuric acid, and the like, preferably at a temperature in the range of about 60° C. to about 120° C., more preferably at about 100° C., to yield the corresponding compound of formula (Ia).

Compounds of formula (I) wherein X is NH and Y is O may be prepared according to the process outlined in Scheme 2.

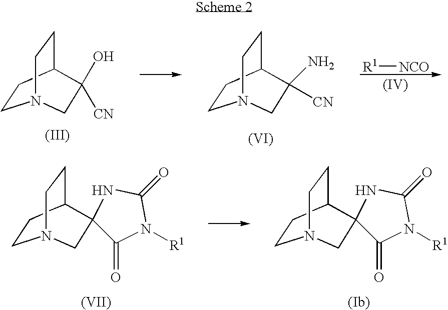

Accordingly, a suitably substituted compound of formula (III) is reacted with a source of ammonia such as ammonium hydroxide, ammonia gas, and the like, in a polar organic solvent such as ethanol, methanol, dioxane, and the like, at a temperature greater than room temperature, preferably at a temperature of about 50° C., to yield the corresponding compound of formula (VI).

The compound of formula (V) is reacted with a suitably substituted isocyanate derivative, a compound of formula (III), a known compound or compound prepared by known methods, preferably in the presence of a catalyst such as CuCl, CuBr, CuI, in an organic solvent such as DMF, DMSO, and the like, preferably in a polar organic solvent which at least partially dissolves the catalyst, at about room temperature, to yield the corresponding compound of formula (VII).

The compound of formula (VII) is reacted with an acid such as concentrated HCl, sulfonic acid, sulfuric acid, and the like, preferably at a temperature in the range of about 60° C. to about 120° C., more preferably at about 100° C., to yield the corresponding compound of formula (Ib).

The compound of formula (Ib) may optionally be reacted according to known methods, (for example by reacting the compound of formula (Ib) with a base and then with a suitably substituted electrophile) to yield the corresponding compound of formula (I) wherein X is NR^{A} and R^{A} is other than hydrogen.

Compounds of formula (I) wherein X is NH and Y is NH may be prepared according to the process outlined in Scheme 3.

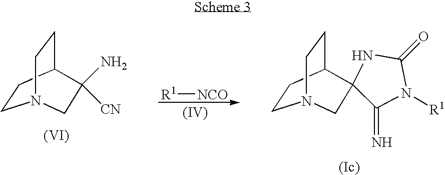

Accordingly, a suitably substituted compound of formula (VI) is reacted with a suitably substituted isocyanate derivative, a compound of formula (III), preferably in the presence of a catalyst such as CuCl, CuBr, CuI, in an organic solvent such as DMF, DMSO, and the like, preferably in a polar organic solvent which at least partially dissolves the catalyst, at about room temperature, to yield the corresponding compound of formula (Ic).

Compounds of formula (II) wherein X is O and Y is NH may be prepared according to the process outlined in Scheme 4.

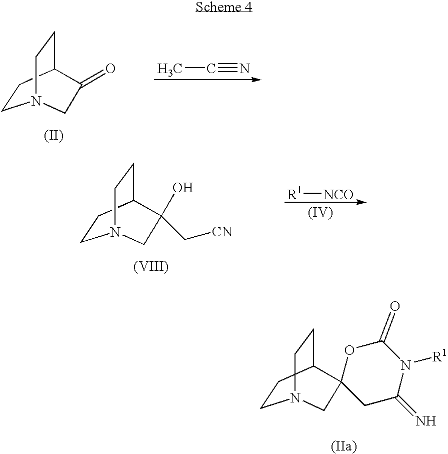

Accordingly, the compound of formula (II), a known compound or compound prepared by known methods is reacted with acetonitrile which has been de-protonated with a base such as n-butyl lithium, t-butyl lithium, sodium hydride, lithium diisopropyl amine, and the like, in an organic solvent such as THF, acetonitrile, and the like, and the like, wherein the reactants are combined at a temperature less than about room temperature, preferably at about −78° C., and then the reaction is completed at room temperature, to yield the corresponding compound of formula (VIII).

The compound of formula (VIII) is reacted with a suitably substituted isocyanate derivative, a compound of formula (III), a known compound or compound prepared by known methods, preferably in the presence of a catalyst such as CuCl, CuBr, CuI, in an organic solvent such as DMF, DMSO, and the like, preferably in a polar organic solvent which at least partially dissolves the catalyst, at about room temperature, to yield the corresponding compound of formula (IIa).

The present invention further comprises pharmaceutical compositions containing one or more compounds of formula (I) and/or compounds of formula (II) with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.1-500 mg and may be given at a dosage of from about 0.1-100 mg/kg/day, preferably from about 0.5-100 mg/kg/day, more preferably from about 1.0-50 mg/kg/day. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method of treating central nervous system disorders described in the present invention may also be carried out using a pharmaceutical composition comprising any of the compounds as defined herein and a pharmaceutically acceptable carrier. The pharmaceutical composition may contain between about 0.1 mg and 500 mg, preferably about 5 to 100 mg, of the compound, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixers, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

The compound of the present invention can also be administered in the form of liposome delivery systems, such as small unilamellar vesicles, large unilamellar vesicles, and multilamellar vesicles. Liposomes can be formed from a variety of phospholipids, such as cholesterol, stearylamine or phophatidylcholines.

Compounds of the present invention may also be delivered by the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropylmethacrylamidephenol, polyhydroxy-ethylaspartamidephenol, or polyethyl eneoxidepolylysine substituted with palmitoyl residue. Furthermore, the compounds of the present invention may be coupled to a class of biodegradable polymers useful in achieving controlled release of a drug, for example, polylactic acid, polyepsilon caprolactone, polyhydroxy butyeric acid, polyorthoesters, polyacetals, polydihydropyrans, polycyanoacrylates and cross-linked or amphipathic block copolymers of hydrogels.

Compounds of this invention may be administered in any of the foregoing compositions and according to dosage regimens established in the art whenever treatment of central nervous system disorders is required.

The daily dosage of the products may be varied over a wide range from 0.01 to 1000 mg per adult human per day. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250 and 500 milligrams of the active ingredient for the symptomatic adjustment of the dosage to the patient to be treated. An effective amount of the drug is ordinarily supplied at a dosage level of from about 0.1 mg/kg to about 500 mg/kg of body weight per day. Preferably, the range is from about 0.1 to about 100 mg/kg of body weight per day, more preferably, from about 0.5 to about 100 mg/kg of body weight per day, more preferably, from about 1.0 to about 50 mg/kg of body weight per day. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

The following Examples are set forth to aid in the understanding of the invention, and are not intended and should not be construed to limit in any way the invention set forth in the claims which follow thereafter.

In the Examples which follow, some synthesis products are listed as having been isolated as a residue. It will be understood by one of ordinary skill in the art that the term “residue” does not limit the physical state in which the product was isolated and may include, for example, a solid, an oil, a foam, a gum, a syrup, and the like.

### EXAMPLE 1

### 3′-(3-trifluoromethylphenyl)-guinuclidine-3-spiro-5′-oxazolidine-2′,4′-dione trifluoroacetate salt (Combound #1)

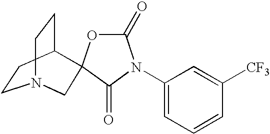 Step A: 3-Hydroxyquinuclidine-3-carbonitrile (following the procedure described in J. Heterocyclic Chem.,18, 1507(1981))

To a stirring solution of quinuclidine hydrochloride (60.22 g, 0.373 mole) in water (78 ml), cooled to 0° C., was added dropwise a solution of potassium cyanide (24.26 g, 0.373 mole) in water (78 ml). After stirring the reaction mixture for three hours at 0° C., the precipitate was filtered, washed with water and dried, to yield the title compound as a white solid.

MS (ESI): 153.19 (M+H⁺).

Step B: 3′-(3-Trifluoromethylphenyl)-quinuclidine-3-spiro-5′-oxazolidine-2′,4′-dione trifluoroacetate.

To a stirring solution of the compound of prepared in Step A above, (0.100 g, 0.66 mmol) and CuCl (0.050 g, 0.50 mmol) in DMF (0.5 ml) at room temperature, was added 3-trifluoromethylphenyl isocyanate (0.094 g, 0.5 mmol, 0.094 ml). After stirring the reaction mixture for 20 min., the reaction mixture was heated at 100° C. for 1 h. Aqueous hydrochloride solution (0.2 ml, 6 N) was then added dropwise and the reaction mixture stirred at 100° C. for another 30 min. After cooling the reaction mixture to room temperature, the reaction mixture was filtered and directly purified via Gilson HPLC purification, to yield the title compound as white solid.

MS (ESI): 341.2 (M+H⁺).

### EXAMPLE 2

### 3′-(4-methylphenyl)-guinuclidine-3-spiro-5′-oxazolidine-2′,4′-dione trifluoroacetate salt (Compound #2)

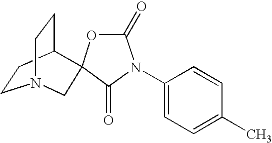

Following the procedure of Example 1, Step B, with substitution of 4-methylphenyl isocyanate for 3-trifluoromethylphenyl isocyanate, the title compound was prepared as a white solid.

MS (ES+) 287.2 [M+H]⁺

### EXAMPLE 3

### 3′-(4-methoxyphenyl)-guinuclidine-3-spiro-5′-oxazolidi ne-2′,4′-dione trifluoroacetate salt (Compound #3)

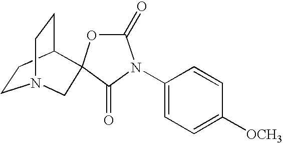

Following the procedure of Example 1, Step B, with substitution of 4-methoxyphenyl isocyanate for 3-trifluoromethylphenyl isocyanate, the title compound was prepared as a white solid.

MS (ES+) 303.2 [M+H]⁺

### EXAMPLE 4

### 3′-(4-methoxy-2-methylphenyl)-guinuclidine-3-spiro-5′-oxazoli-dine-2′,4′-dione trifluoroacetate salt (Compound #4)

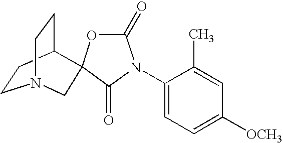

Following the procedure of Example 1, Step B, with substitution of 4-methoxy-2-methylphenyl isocyanate for 3-trifluoromethylphenyl isocyanate, the title compound was prepared as a white solid.

MS (ES+) 317.2 [M+H]⁺

### EXAMPLE 5

### Preparation of 3′-(3,4-dichlorophenyl)-guinuclidine-3-spiro-5′-oxazolidine-2′,4′-dione trifluoroacetate salt (Compound #5)

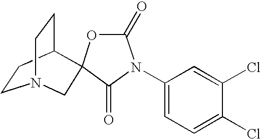

Following the procedure of Example 1, Step B, with substitution of 3,4-dichlorophenyl isocyanate for 3-trifluoromethylphenyl isocyanate, the title compound was prepared as a white solid.

MS (ES+) 341.1 [M+H]⁺

### EXAMPLE 6

### 3′-(3-nitrophenyl)-guinuclidine-3-spiro-5′-oxazolidine-2′,4′-dione trifluoroacetate salt (Compound #6)

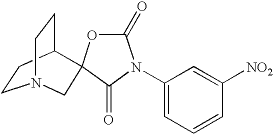

Following the procedure of Example 1, Step B, with substitution of 3-nitrophenyl isocyanate for 3-trifluoromethylphenyl isocyanate, the title compound was prepared as a white solid.

MS (ES+) 318.2 [M+H]⁺

### EXAMPLE 7

### 3′-(3,5-dichlorophenvi)-guinuclidine-3-spiro-5′-oxazolidine-2′,4′-dione trifluoroacetate salt (Compound #7)

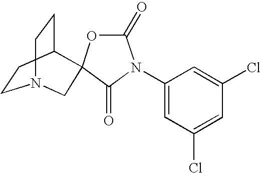

Following the procedure of Example 1, Step B, with substitution of 3,5-dichlorophenyl isocyanate for 3-trifluoromethylphenyl isocyanate, the title compound was prepared as a white solid.

MS (ES+) 341.1 [M+H]⁺

### EXAMPLE 8

### 3′-(3,5-bis(trifluoromethyl)phenyl)-guinuclidine-3-spiro-5′-oxazolidine-2, 4′-dione trifluoroacetate salt (Compound #8)

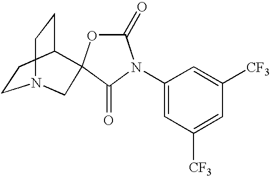

Following the procedure of Example 1, Step B, with substitution of 3,5-bis(trifluoromethyl)phenyl isocyanate for 3-trifluoromethylphenyl isocyanate, the title compound was prepared as a white solid.

MS (ES+) 409.2 [M+H]⁺

### EXAMPLE 9

### 3′-(2-fluorophenvl)-guinuclidine-3-spiro-5′-oxazolidine-2′,4′-dione trifluoroacetate salt (Compound #9)

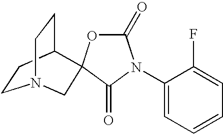

Following the procedure of Example 1, Step B, with substitution of 2-fluorophenyl isocyanate for 3-trifluoromethylphenyl isocyanate, the title compound was prepared as a white solid.

MS (ES+) 291.2 [M+H]⁺

### EXAMPLE 10

### 3′-(3-chlorophenyl)-guinuclidine-3-spiro-5′-oxazolidine-2′,4′-dione trifluoroacetate salt (Compound #10)

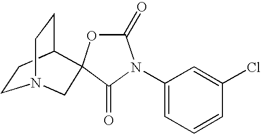

Following the procedure of Example 1, Step B, with substitution of 3-chlorophenyl isocyanate for 3-trifluoromethylphenyl isocyanate, the title compound was prepared as a white solid.

MS (ES+) 307.1 [M+H]⁺

### EXAMPLE 11

### 3′-(2.5-dimethyphenyl)-guinuclidine-3-spiro-5′-oxazolidine-2′,4′-dione trifluoroacetate salt (Compound #11)

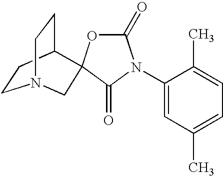

Following the procedure of Example 1, Step B, with substitution of 2,5-dimethylphenyl isocyanate for 3-trifluoromethylphenyl isocyanate, the title compound was prepared as a white solid.

MS (ES+) 301.2 [M+H]⁺

### EXAMPLE 12

### 3′-(2.5-dichlorophenyl)-guinuclidine-3-spiro-5′-oxazolidine-2′,4′-dione trifluoroacetate salt (Compound #12)

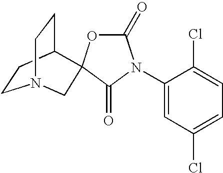

Following the procedure of Example 1, Step B, with substitution of 2,5-dichlorophenyl isocyanate for 3-trifluoromethylphenyl isocyanate, the title compound was prepared as a white solid.

MS (ES+) 341.1 [M+H]⁺

### EXAMPLE 13

### 3-(3-ethoxycarbonylphenyl)-guinuclidine-3-spiro-5′-oxazolidine-2′,4′-dione trifluoroacetate salt (Compound #13)

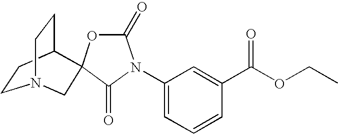

Following the procedure of Example 1, Step B, with substitution of 3-ethoxycarbonylphenyl isocyanate for 3-trifluoromethylphenyl isocyanate, the title compound was prepared as a white solid.

MS (ES+) 345.2 [M+H]⁺

### EXAMPLE 14

### 3′-(3-methoxyphenyl)-guinuclidine-3-spiro-5′-oxazolidine-2, 4′-dione trifluoroacetate salt (Compound #14)

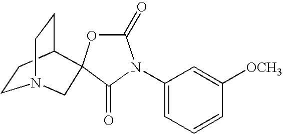

Following the procedure of Example 1, Step B, with substitution of 3-methoxyphenyl isocyanate for 3-trifluoromethylphenyl isocyanate, the title compound was prepared as a white solid.

MS (ES+) 303.2 [M+H]⁺

### EXAMPLE 15

### 3′-(3-hydroxyphenyl)-guinuclidine-3-spiro-5′-oxazolidine-2′,4′-dione trifluoroacetate salt (Compound #15)

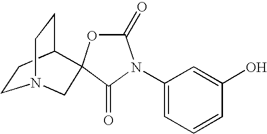

To a stirring solution of the compound prepared as in Example 14 (0.086 g, 0.20 mmole) in methylene chloride (2.0 ml), cooled at −70° C., was added dropwise boron tribromide (1.0 mmole, 1 M in methylene chloride, 1.0 ml). After stirring the reaction mixture at room temperature overnight, methanol (1.0 ml) was added. The solvent was removed and the resulting residue was purified by Gilson HPLC, to yield the title compound as a white solid.

MS (ES+) 289.2 [M+H]⁺

### EXAMPLE 16

### 3′-(3.4-dimethylphenyl)-guinuclidine-3-spiro-5′-oxazolidine-2′,4′-dione trifluoroacetate salt (Compound #16)

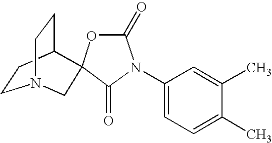

Following the procedure of Example 1, Step B, with substitution of 3,4-dimethylphenyl isocyanate for 3-trifluoromethylphenyl isocyanate, the title compound was prepared as a white solid.

MS (ES+) 301.2 [M+H]+

### EXAMPLE 17

### 3′-(4-hydroxyphenyl)-guinuclidine-3-spiro-5′-oxazolidine-2′,4′-dione trifluoroacetate salt (Compound #18)

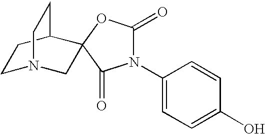

Following the procedure of Example 15, with substitution of the compound prepared as in Example 3 for the compound prepared as in Example 14, the title compound was prepared as a white solid.

MS (ES+) 289.2 [M+H]+

### EXAMPLE 18

### 3′-(4-hydroxy-2-methylphenyl)-quinuclidine-3-spiro-5′-oxazolidine-2′,4′-dione trifluoroacetate salt (Compound #19)

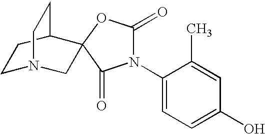

Following the procedure of Example 15, with substitution of the compound prepared as in Example 4 for the compound prepared as in Example 14, the title compound was prepared as a white solid.

MS (ES+) 303.2 [M+H]+

### EXAMPLE 19

### 3′-(3,5-dimethylphenyl)-guinuclidine-3-spiro-5′-oxazolidine-2′,4′-dione trifluoroacetate salt (Compound #20)

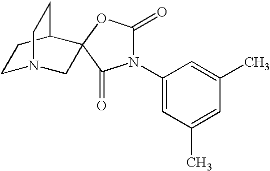

Following the procedure of Example 1, Step B, with substitution of 3,5-dimethylphenyl isocyanate for 3-trifluoromethylphenyl isocyanate, the title compound was prepared as a white solid.

MS (ES+) 301.2 [M+H]+

### EXAMPLE 20

### 3′-(2,4-difluorophenyl)-guinuclidine-3-spiro-5′-oxazolidine-2′,4′-dione trifluoroacetate salt (Compound #21)

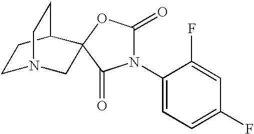

Following the procedure of Example 1, Step B, with substitution of 2,4-difluorophenyl isocyanate for 3-trifluoromethylphenyl isocyanate, the title compound was prepared as a white solid.

MS (ES+) 309.2 [M+H]+

### EXAMPLE 21

### 3′-(2,4-dimethoxyphenyl)-quinuclidine-3-spiro-5′-oxazolidine-2′,4′-dione trifluoroacetate salt (Compound #23) t,0342

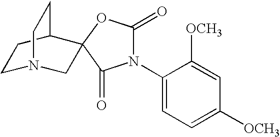

Following the procedure of Example 1, Step B, with substitution of 2, 4-dimethoxyphenyl isocyanate for 3-trifluoromethylphenyl isocyanate, the title compound was prepared as a white solid.

MS (ES+) 333.2 [M+H]+

### EXAMPLE 22

### 3′-(3-chloro-4-methylphenv)-guinuclidine-3-spiro-5′-oxazolidine-2′, 4′-dione trifluoroacetate salt (Compounds #24)

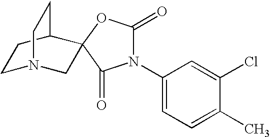

Following the procedure of Example 1, Step B, with substitution of 3-chloro-4-methylphenyl isocyanate for 3-trifluoromethylphenyl isocyanate, the title compound was prepared-as a white solid.

MS (ES+) 301.2 [M+H]+

### EXAMPLE 23

### 3′-(3-fluorophenyl)-guinuclidine-3-spiro-5′-oxazolidine-2′,4′-dione trifluoroacetate salt (Compound #25)

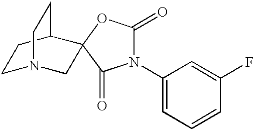

Following the procedure of Example 1, Step B, with substitution of 3-fluorophenyl isocyanate for 3-trifluoromethylphenyl isocyanate, the title compound was prepared as a white solid.

MS (ES+) 291.2 [M+H]+

### EXAMPLE 24

### 3′-(2-methylphenyl)-guinuclidine-3-spiro-5′-oxazolidine-2′,4′-dione trifluoroacetate salt (Compound #26)

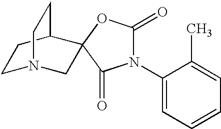

Following the procedure of Example 1, Step B, with substitution of 2-methylphenyl isocyanate for 3-trifluoromethylphenyl isocyanate, the title compound was prepared as a white solid.

MS (ES+) 287.2 [M+H]+

### EXAMPLE 25

### 3′-(2,3-dimethylphenyl)-quinuclidine-3-spiro-5′-oxazolidine-2′,4′-dione trifluoroacetate salt (Compound #27)

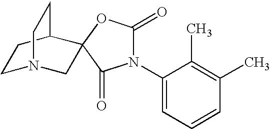

Following the procedure of Example 1, Step B, with substitution of 2,3-dimethylphenyl isocyanate for 3-trifluoromethylphenyl isocyanate, the title compound was prepared as a white solid.

MS (ES+) 301.2 [M+H]+

### EXAMPLE 26

### 3′-(2-isopropylphenyl)-quinuclidine-3-spi ro-5′-oxazolidine-2′,4′-dione trifluoroacetate salt (Compound #28)

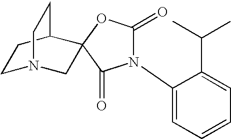

Following the procedure of Example 1, Step B, with substitution of 2-isopropylphenyl isocyanate for 3-trifluoromethylphenyl isocyanate, the title compound was prepared as a white solid.

MS (ES+) 315.2 [M+H]+

### EXAMPLE 27

### 3′-(2,5-dimethoxyphenyl)-quinuclidine-3-spiro-5′-oxazolidine-2′,4′-dione trifluoroacetate salt (Compound #29)

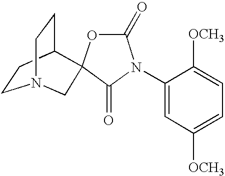

Following the procedure of Example 1, Step B, with substitution of 2, 5-dimethoxyphenyl isocyanate for 3-trifluoromethylphenyl isocyanate, the title compound was prepared as a white solid.

MS (ES+) 333.2 [M+H]+

### EXAMPLE 28

### 3′-(2-ethoxycarbonvlmethyl)-quinuclidine-3-spiro-5′-oxazolidine-2′,4′-dione hydrochloride (Compound #30)

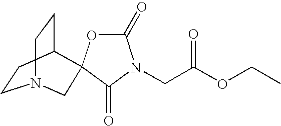

To a stirring solution of the compound of prepared as in Example 1, Step A (3.19 g, 25 mmol) and CuCl (2.52 g, 25 mmol) in DMF (40 ml) at room temperature, was added ethyl 2-isocyanatoacetate(2.9 ml, 25 mmol). After 20 min. of stirring, the solution was heated at 100° C. for 1 h. After cooling down to room temperature, the crude product was precipitated by adding water and purified by flash chromatography on 230-400 mesh silica gel, eluting with 95:5 of CHCl3/CH₃OH. The product was recrystallized from a mixture of ethanol, hydrochloric acid and acetone, to yield the title compound as yellow solid.

MS (ESI): 283.2 (M+H+).

### EXAMPLE 29

### 3′-(3-trifludromethylphenyl)-quinuclidine-3-spiro-5′-hydantoins trifluoroacetate salt (Compound #40)

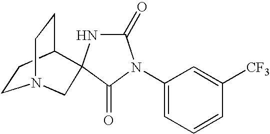 Step A: 3-Aminoquinuclidine-3-carbonitrile(Synthesis, 1994, 832-836)

To a stirring solution of the compound prepared as in Example 1, Step A, (8.0 g, 52 mole) in ethanol (40 ml), was added dropwise a solution of ammonia hydroxide (28%, 6.0 ml, 48 mmol). The resulting solution was heated at 50° C. for 72 hrs and then stirred at room temperature for another 12 hrs. After removing the solvent by roto-evaporator, the residue was purified by recrystallization in ethanol to yield the title compound as a white solid.

MS (ESI): 152.2 (M+H+).

¹H NMR (300 MHz, CD₃OD), δ: 3.86(d, J=4 Hz, 1H), 3.60-3.30(m, 5H), 2.84-2.67(m, 2H), 2.53-2.40(m, 2H), 2.11-2.00 (m,1 H).

Step B: 3′-(3-Trifluoromethylphenyl)-quinuclidine-3-spiro-5′-hydantoins trifluoroacetate.

To a stirring solution of the compound of prepared as in Step A above, (0.10 g, 0.65 mmol) and CuCl (0.065 g, 0.65 mmol) in DMF (0.5 ml) at room temperature, was added 3-trifluoromethylphenyl isocyanate (0.147 g, 0.78 mmole). After stirring 18 hrs at room temperature, hydrochloric acid (6N, 0.25 ml) was added. The solution was heated at 100° C. for 1 h. After cooling down to room temperature, the solution was filtered and purified by Gilson HPLC, to yield the title compound as white solid.

MS (ESI): 340.2 (M+H+)

¹H NMR (300 MHz, DMSO), δ: 9.33(s, 1H), 7.86(s, 1H), 7.79-7.74(m, 3H), 3.66(d, j=4 Hz, 1H), 3.40-3.10(m, 5H), 2.50-2.46(m, 2H), 2.30-2.10(m, 1H), 1.90-1.80 (m, 2H).

### EXAMPLE 30

### 3′-(1-naphthvl)-quinuclidine-3-spiro-5′-hydantoins trifluoroacetate salt (Compound #41)

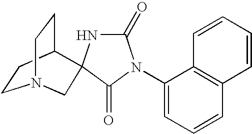

Following the procedure of Example 29, Step B, with substitution of 1-naphthyl isocyanate for 3-trifluoromethylphenyl isocyanate, the title compound was prepared as a white solid.

MS (ES+) 322.2 [M+H]+

### EXAMPLE 31

### 3′-(4-trifluoromethylphenyl)-quinuclidine-3-spiro-5′-hydantoins trifluoroacetate salt (Compound #42)

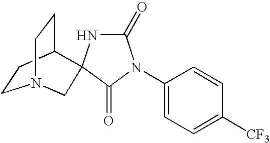

Following the procedure of Example 29, Step B, with substitution of 4-trifluoromethylphenyl isocyanate for 3-trifluoromethylphenyl isocyanate, the title compound was prepared as a white solid.

MS (ES+) 340.2 [M+H]+

### EXAMPLE 32

### 3′-(3,5-bis(methoxycarbonyl)phenvi)-quinuclidine-3-spiro-5′-hydantoins trifluoroacetate salt (Compound #43)

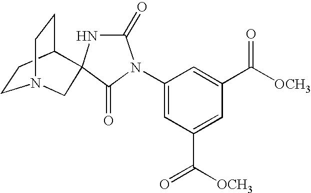

Following the procedure of Example 29, Step B, with substitution of 3,5-bis(methoxycarbonyl)phenyl isocyanate for 3-trifluoromethylphenyl isocyanate, the title compound was prepared as a white solid.

MS (ES+) 388.2 [M+H]+

### EXAMPLE 33

### 3′-(3,5-bis(trifluromethyl)phenyl)-quinuclidine-3-spiro-5′-hydantoins trifluoroacetate salt (Compound #44)

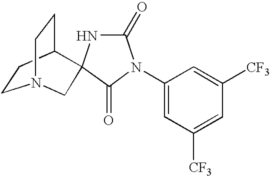

Following the procedure of Example 29, Step B, with substitution of 3,5-bis(trifluoromethyl)phenyl isocyanate for 3-trifluoromethylphenyl isocyanate, the title compound was prepared as a white solid.

MS (ES+) 408.2 [M+H]+

### EXAMPLE 34

### 3′-(2-fluorophenyl)-quinuclidine-3-sgiro-5′-hydantoins trifluoroacetate salt (Compound #45)

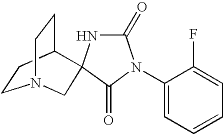

Following the procedure of Example 29, Step B, with substitution of 2-fluorophenyl isocyanate for 3-trifluoromethylphenyl isocyanate, the title compound was prepared as a white solid.

MS (ES+) 290.2 [M+H]+

### EXAMPLE 35

### 3′-(3-ethoxycarbonylphenyl)-quinuclidine-3-sgiro-5′-hydantoins trifluoroacetate salt (Compound #46)

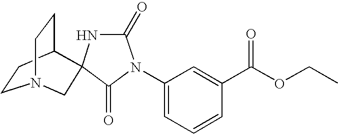

Following the procedure of Example 29, Step B, with substitution of 3-ethoxycarbonylphenyl isocyanate for 3-trifluoromethylphenyl isocyanate, the title compound was prepared as a white solid.

MS (ES+) 344.2 [M+H]+

### EXAMPLE 36

### 3′-(3,4-dimethylphenyl)-quinuclidine-3-spiro-5′-hydantoins trifluoroacetate salt (Compound #47)

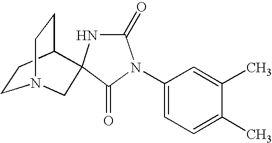

Following the procedure of Example 29, Step B, with substitution of 3,4-dimethylphenyl isocyanate for 3-trifluoromethylphenyl isocyanate, the title compound was prepared as a white solid.

MS (ES+) 300.2 [M+H]+

### EXAMPLE 37

### 3′-(3.4-dichlorophenyl)-quinuclidine-3-spiro-5′-hvdantoins trifluoroacetate salt (Compound #48)

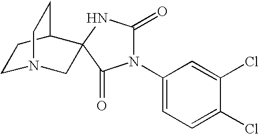

Following the procedure of Example 29, Step B, with substitution of 3,4-dichlorophenyl isocyanate for 3-trifluoromethylphenyl isocyanate, the title compound was prepared as a white solid.

MS (ES+) 340.1 [M+H]+

### EXAMPLE 38

### 3′-(2,4-dimethoxyphenyl)-quinuclidine-3-spiro-5′-hvdantoins trifluoroacetate salt (Compound #49)

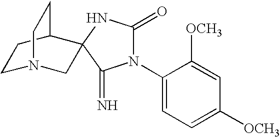

To a stirring solution of the compound prepared as in Example 29, Step A, (0.20 g, 1.3 mmol) and CuCl (0.129 g, 1.3 mmol) in DMF (1.0 ml) at room temperature, was added 2,4-dimethoxyphenyl isocyanate (0.279 g, 1.5 mmole). After stirring 24 hrs at room temperature, hydrochloric acid (6N, 0.20 ml) was added. The solution was heated at 1 00C for 30 min. After cooling down to room temperature, the solution was filtered and purified by Gilson HPLC, to give the title compound as purple solid.

MS (ESI): 331.1 (M+H+).

### EXAMPLE 39

### 3′-(4-methoxyphenyl)-quinuclidine-3-spiro-4′-iminooxan-2-ones trifluoroacetate salt (Compound #100).

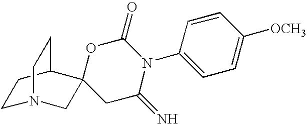 Step A: 3-Hydroxyquinuclidine-3-methylnitrile(RWJ356576-300-A).

To a stirring solution of acetonitrile (3.8 ml, 72.27 mmol) in THF(60 ml) at −78° C., n-butyl lithium (2.0 M in THF, 29 ml, 58 mmol) was added slowly over about 1 hour. After completed addition of the n-butyl lithium, 3-quinuclidinone (6.0 g, 47.9 mmol) in THF (20 ml) was added slowly while the solution temperature was maintained at −78° C. The solution was stirred for another 15 min, then allowed to return to room temperature and maintained at this temperature for two hours. Water (5.0 ml) was added to the reaction mixture terminate the reaction. Sodium bicarbonate (2.0 g) was then added. The solution was then diluted with ethyl acetate, dried over MgSO₄, and filtered. After removing solvent, the residue was purified over silica gel column eluted with a mixture of solvents: methylene chloride / methanol / ammonium hydroxide (at a ratio of 95:5:1) to yield the title compound as a white solid.

MS (ESI): 167.2 (M+H+)

¹H NMR (300 MHz, DMSO), δ: 5.09(s,1 H), 2.55-2.81 (m, 8H), 1.90(m, 1H), 1.75(m, 1H), 1.43-1.56(m, 2H), 1.20-1.30(m, 1H).

Step B: 3′-(4-methoxyphenyl)-quinuclidine-3-spiro-4′-iminooxan-2-ones trifluoroacetate salt

To a stirring solution of the compound prepared as in Step A above, (0.100 g, 0.6 mmol) and copper(I) chloride (0.050 g, 0.5 mmol) in DMF (1.0 ml) at room temperature was added 4-methoxyphenyl isocyanate (0.075 g, 0.5 mmol) and the reaction mixture was stirred at room temperature overnight. Water (0.2 ml) was then added, the solution was filtered and purified by Gilson HPLC, to yield title compound as a white solid.

MS (ESI): 316.3 (M+H+)

¹H NMR (300 MHz, DMSO), δ: 9.85(s, 1H), 7.38(d, J=8 Hz, 2H), 6.88(d, J=8 Hz, 2H), 3.78-3.96(m, 2H), 3.71 (s, 3H), 3.55-3.16(m, 6H), 2.43(m, broad, 1H), 2.19(m, broad, 1H), 1.96 (m, broad, 3H).

### EXAMPLE 40

### 3′-(4-methylphenyl)-quinuclidine-3-spiro-4′-iminooxan-2-ones trifluoroacetate salt (Compound #101)

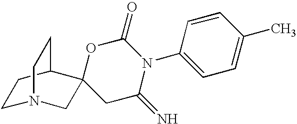

To a stirring solution of the compound prepared as in Example 39, Step A, (0.050 g, 0.3 mmol) and copper(I) chloride (0.025 g, 0.25 mmol) in DMF (0.5 ml) at room temperature was added 4-methylphenyl isocyanate (0.044 g, 0.33 mmol) and the solution stirred at room temperature overnight. Water (0.1 ml) was then added, the solution was filtered and purified by Gilson HPLC to yield title compound as a white solid.

MS (ESI): 300.2 (M+H+).

### EXAMPLE 41

### 3′-(3-methoxyphenyl)-quinuclidine-3-spiro-4′-iminooxan-2-ones trifluoroacetate salt (Compound #102)

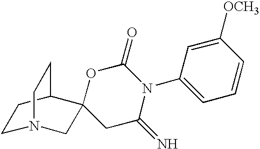

Following the procedure of Example 40, Step B, with substitution of 3-methoxyphenyl isocyanate for 4-methylphenyl isocyanate, the title compound was prepared as a white solid.

MS (ES+) 316.2 [M+H]+

### EXAMPLE 42

### 3′-(3-trifluoromethylphenyl)-quinuclidine-3-spiro-4′-iminooxan-2-ones trifluoroacetate salt (Compound #103)

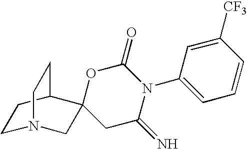

Following the procedure of Example 40, Step B, with substitution of 3-methoxyphenyl isocyanate for 4-methylphenyl isocyanate, the title compound was prepared as a white solid.

MS (ES+) 354.3 [M+H]+

### EXAMPLE 43

### 3′-(3,4-dichlorophenyl)-quinuclidine-3-spiro-4′-iminooxan-2-ones trifluoroacetate salt (Compound #104)

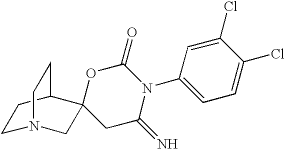

Following the procedure of Example 40, Step B, with substitution of 3,4-dichlorophenyl isocyanate for 4-methylphenyl isocyanate, the title compound was prepared as a white solid.

MS (ES+) 354.2 [M+H]+

### EXAMPLE 44

### 3′-(2,4-dimethoxyphenyl)-quinuclidine-3-spiro-4′-iminooxan-2-ones trifluoroacetate sat (Compound #105)

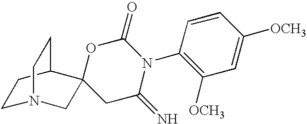

Following the procedure of Example 40, Step B, with substitution of 2,4-dimethoxyphenyl isocyanate for 4-methylphenyl isocyanate, the title compound was prepared as a gray solid.

,MS (ES+) 346.2 [M+H]+

### EXAMPLE 45

### 3′-(3,5-dimethylphenyl)-quinuclidine-3-spiro-4′-iminooxan-2-ones trifluoroacetate salt (Compound #106)

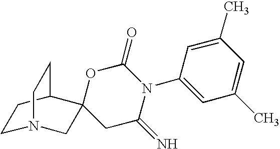

Following the procedure of Example 40, Step B, with substitution of 3,5-dimethylphenyl isocyanate for 4-methylphenyl isocyanate, the title compound was prepared as a white solid.

MS (ES+) 314.3 [M+H]+

### EXAMPLE 46

### 3′-(3,5-dichlorophenyl)-quinuclidine-3-spiro-4′-iminooxan-2-ones trifluoroacetate salt (Compound #107)

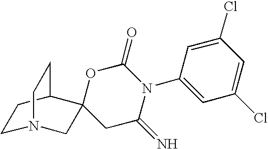

Following the procedure of Example 40, Step B, with substitution of 3,5-dichlorphenyl isocyanate for 4-methylphenyl isocyanate, the title compound was prepared as a white solid.

MS (ES+) 354.2 [M+H]+

### EXAMPLE 47

### 3′-(3-chlorophenyl)-quinuclidine-3-spiro-4′-iminooxan-2-ones trifluoroacetate salt (Compound #108)

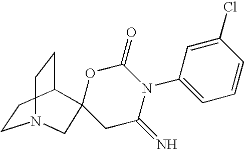

Following the procedure of Example 40, Step B, with substitution of 3-chlorophenyl isocyanate for 4-methylphenyl isocyanate, the title compound was prepared as a white solid.

MS (ES+) 320.2 [M+H]+

### EXAMPLE 48

### 3′-(3-chloro-4-methylphenyl)-quinuclidine-3-spiro-4′-iminooxan-2-ones trifluoroacetate salt (Compound #109)

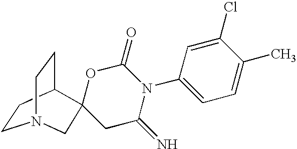

Following the procedure of Example 40, Step B, with substitution of 3-chloro-4-methylphenyl isocyanate for 4-methylphenyl isocyanate, the title compound was prepared as a white solid.

MS (ES+) 334.2 [M+H]+

### EXAMPLE 49

### 3′-(3-ethoxycarbonylphenyl)-quinuclidine-3-spiro-4′-iminooxan-2-ones trifluoroacetate salt (Compound #110)

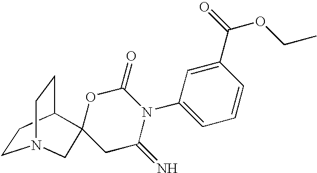

Following the procedure of Example 40, Step B, with substitution of 3-ethoxycarbonylphenyl isocyanate for 4-methylphenyl isocyanate, the title compound was prepared as a white solid.

MS (ES+) 358.3 [M+H]+

### EXAMPLE 50

### 3′-(3.5-dimethoxyphenyl)-quinuclidine-3-spiro-4′-iminooxan-2-ones trifluoroacetate salt (Compound #111)

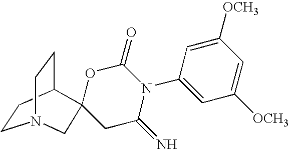

Following the procedure of Example 40, Step B, with substitution of 3,5-dimethoxyphenyl isocyanate for 4-methylphenyl isocyanate, the title compound was prepared as a white solid.

MS (ES+) 346.3 [M+H]+

### EXAMPLE 51

### 3′-phenyl-quinuclidine-3-spiro-4′-iminooxan-2-ones trifluoroacetate salt (Compound #112)

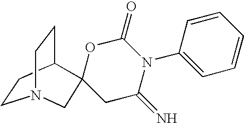

Following the procedure of Example 40, Step B, with substitution of phenyl isocyanate for 4-methylphenyl isocyanate, the title compound was prepared as a white solid.

MS (ES+) 286.1 [M+H]+

### EXAMPLE 52

### 3′-(2-methyl-4-methoxyphenyl)quinuclidine-3-spiro-4′-iminooxan-2-ones trifluoroacetate salt (Compound #113)

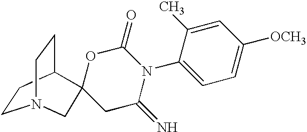

Following the procedure of Example 40, Step B, with substitution of 2-methyl-4-methoxyphenyl isocyanate for 4-methylphenyl isocyanate, the title compound was prepared as a black solid.

MS (ES+) 330.1 [M+H]+

### EXAMPLE 53

### 3′-(2-methoxyphenyl)quinuclidine-3-spiro-4′-iminooxan-2-ones trifluoroacetate salt (Compound #144)

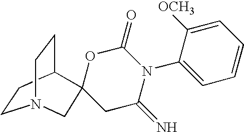

Following the procedure of Example 40, Step B, with substitution of 2-methoxyphenyl isocyanate for 4-methylphenyl isocyanate, the title compound was prepared as black oil.

MS (ES+) 316.1 [M+H]+

### EXAMPLE 54

### 3′-(2,4-difluorophenyl)quinuclidine-3-spiro-4′-iminooxan-2-ones trifluoroacetate salt (Compound 115)

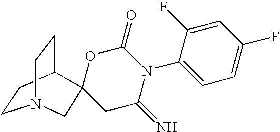

Following the procedure of Example 40, Step B, with substitution of 2, 4-difluorophenyl isocyanate for 4-methylphenyl isocyanate, the title compound was prepared as colorless oil.

MS (ES+) 322.0 [M+H]+

### EXAMPLE 55

### Aβ Aggregation Assay

Aβs are known to form aggregates leading to the formation of amyloid plaques that are characteristic of Alzheimer's disease. This phenomenon can be demonstrated in vitro by using Synthaloid plates, coated with Aβ crystallization centers, and labelled Aβs for detecting aggregation.

This aggregation assay was validated using both ¹²⁵I-Aβ,₁₋₄₀ and fluo-Aβ₁₋₄₀ in a buffer containing 50 mM HEPES, pH 7.4, 0.1% BSA, 10% FCS and protease inhibitors.

To each well of a 96-well Synthaloid plate was added 100 nM of fluo-Aβ₁₋₄₀ in 100μl. To each well was then added 10 μM of test compound. The plate was then allowed to incubate for 2.5 hr at room temperature. At the end of the incubation, unbound protein in the wells was removed by three washes using the buffer as described above. The amount of bound protein (which represents the amount of Aβ aggregation) was measured either by fluorescence measurements.

Higher fluorescence values relative to vehicle wells were interpreted as an indication of greater Aβ aggregation. Thus a decrease in fluorescence for the test compound well relative to the vehicle well indicated that the test compound prevents aggregation to some extent. Higher % values in the data in Table 4 below, below indicates greater effectiveness at preventing aggregation.

### EXAMPLE 56

### % Inhibition of AcH Release Assay

Synaptosomes from guinea pig hippocampus were incubated with 0.1 μM ³H-choline and then subjected to repeated washes with buffer to remove unincorporated ³H-choline. The synaptosomes were then treated with 65 mM K⁺for 30 seconds to elicit 3H-acetylcholine release.

Both Aβ₁₋₄₀ and Aβ₁₋₄₂ at 100 pM are known to inhibited acetylcholine release from these preparations (33% inhibition for both Aβ₁₋₄₀ and Aβ₁₋₄₂). To determine if a test compound will inhibit acetylcholine release, the synaptosomes were pre-treated with 10 μM of the test compound prior to the K+ stimulation. The decrease or increase in acetylcholine release was then measured by superfusion.

Both the Aβ aggregation assay as described in Example 55, particularly as run using the ¹²⁵I-Aβ₁₋₄₀, and the % inhibition of AcH release assay as described in Example 56, were highly variable on a run-to-run basis. However, the relative activities of the tested compounds were reproducible.

Representative compounds of the present invention were tested according to the procedure described In Example 55 and 56 above, with results as listed in Table 4.

TABLE 4

| ID No. | Aβ Aggregation (%) | AcH Release (%) |
|---|---|---|
| 1 | 100 | 36 |
| 2 | 44 | 74 |
| 3 | 100 | 27 |
| 4 | 95 | 36 |
| 5 | 98 | 27 |
| 6 | 21 | 64 |
| 7 | 62 | 70 |
| 8 | 0 | 77 |
| 9 | 100 | 16 |
| 10 | 50 | 60 |
| 11 | 0 | 71 |
| 12 | 85 | 17 |
| 13 | 100 | 20 |
| 14 | 42 | 14 |
| 15 | 50 | 55 |
| 16 | 67 | 44 |
| 18 | 0 | 23 |
| 19 | 0 | 27 |
| 20 | 0 | 53 |
| 21 | 1 | 17 |
| 24 | 40 | 44 |
| 25 | 5 | 48 |
| 26 | 45 | 54 |
| 27 | 35 | 25 |
| 28 | 0 | 47 |
| 29 | 100 | 20 |
| 30 | 100 | 21 |
| 100 | 48 | 34 |
| 101 | 27 | 24 |
| 104 | 0 | 43 |
| 106 | 80 | 18 |
| 107 | 30 | 54 |
| 108 | 70 | 63 |
| 109 | 80 | 33 |

### EXAMPLE 58

### M1 Muscarinic Receptor % Inhibition Assay

The reagents and materials used in this assay were as follows: 50 mM Tris-HCl, 10 mM MgCl₂, 1 mM EDTA, 0.375% BSA, pH 7.4 buffer; M1 human CHO receptor membranes (Amersham 6110503, 200 Units): 9.7 μg/μl, vials of 1 ml; WGA SPA Beads (Amersham SPQ0031, 12.5 grams): vials of 500 mg; Tritiated Scoplamine Ligand (Perkin Elmer NET-636 250 μCi, 250 μl): 12 μM, vials of 250 μl; 30% DMSO in 50 mM Hepes pH 7.4; Assay Plates (Dynatech 96 well white flat bottom) and Seals (Clear- Zymark #74845, 3 rolls of 2000 seals)

The ligand was prepared as 5nM 48 μl 12 μM+115 ml buffer (900 μl per well to a 2 ml deepwell). The membrane (4.54 mg/ml) and bead (0.03 μg/μl) complex was prepared as 4 vials of beads (500 mg/vial)+40 ml buffer (10 ml per bottle)+1.36 ml membrane (9.7 μg/μl)+400 ml buffer (final volume=440 ml). Test compounds were diluted to 0.4 mM (30% DMSO in 50 mM HEPES, pH 7.4). The positive control was atropine.

The HTS assay was run as follows: SPA-beads were pre-coupled with membrane in binding buffer for at least 30 min at room temperature. 75 μL of buffer was added to each well of a Dynateh 96 well white flat bottom plates with 200 μl final volume. To each well was then added 5 μl of test compound or control (atropine). To each well was then added 20 μl of ligand and 100 μl of the membrane and bead complex. The plate was then incubated for 60 minutes at room temperature. The plate was clear sealed and centrifuged at 1200-1500 rpm for 5 min using a Beckman, Allegra 6K-R centrifuge. Each well was then measured by counting for 1 minute per well on TopCount with 1 min pre-read delay. The results were then analyzed as follows: Nonspecific binding (NSB) =the mean of counts per minute (CPM) of the 10 μM Atropine wells 12E - 12H; Total Binding (TB)=the mean of the wells with no Atropine 12A - 12D; % Inhibition for the test compounds=[1-(Sample CPM - NSB)×100]/TB-NSB

Compounds which inhibit the M1 muscarinic receptor are useful for the treatment of for example, mild cognitive impairment, Alzheimer's disease, dementia, senility and the like.

Compound #109 was tested according to the procedure as described above with the following measured activity: & Inhibition © 1 μM=90%, % Inhibition © 10μM=98%.

### EXAMPLE 59

As a specific embodiment of an oral composition, 100 mg of the Compound #1 is formulated with sufficient finely divided lactose to provide a total amount of 580 to 590 mg to fill a size 0 hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

We Claim:

1. The present invention is directed to a compound of formula (I) 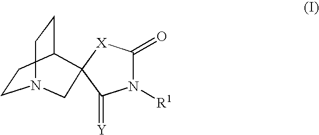
wherein
X is selected from the group consisting of O and NR^{A}; and Y is selected from the group consisting of O and NR^{B}; wherein R^{A} and R^{B} are each selected from the group consisting of hydrogen and lower alkyl;
provided that when X is O, then Y is O;
provided that when X is NR^{A} and Y is NR^{B} then R^{A} and R^{B} are each hydrogen;
R¹ is selected from the group consisting of alkyl, alkoxycarbonyl and aryl; wherein the aryl is optionally substituted with one or more substituents independently selected from halogen, hydroxy, carboxy, alkyl, alkoxy, nitro, cyano, NR^{C}R^{D}, halogenated lower alkyl, halogenated lower alkoxy, alkoxycarbonyl or —S(O)₀₋₂-alkyl;
wherein each R^{c} and R^{D} is independently selected from hydrogen or lower alkyl;
provided that when X is NH and Y is O, then R¹ is other than methyl;
or a pharmaceutically acceptable salt thereof.

2. A compound as in claim 1, wherein
X is selected from the group consisting of O and NR^{A}; and Y is selected from the group consisting of o and NR^{B}; wherein R^{A} and R^{B} are each selected from the group consisting of hydrogen and lower alkyl;
provided that when X is O, then Y is O;
provided that when X is NR^{A} and Y is NR^{B} then R^{A} and R^{B} are each hydrogen;
R¹ is selected from the group consisting of lower alkyl, lower alkoxycarbonyl-(lower alkyl), aryl and substituted aryl; wherein the substituents on the aryl group are one to two independently selected from hydroxy, halogen, lower alkyl, lower alkoxy, trifluoromethyl, nitro, cyano, lower alkoxycarbonyl, lower alkylthio or lower alkylsulfonyl.
provided that when X is NH and Y is O, then R¹ is other than methyl;
or a pharmaceutically acceptable salt thereof.

3. A compound as in claim 2, wherein
X is selected from the group consisting of O and NH; and Y is selected from the group consisting of O and NH;
provided that when X is O, then Y is O;
R¹ is selected from the group consisting of lower alkyl, lower alkoxycarbonyl-(lower alkyl), aryl and substituted aryl; wherein the substituents on the aryl are one to two substituents independently selected from hydroxy, halogen, lower alkyl, lower alkoxy, trifluoromethyl, nitro, cyano, lower alkoxycarbonyl, lower alkylthio or lower alkylsulfonyl;
provided that when X is NH and Y is O, then R¹ is other than methyl;
or a pharmaceutically acceptable salt thereof.

4. A compound as in claim 3, wherein
X is selected from the group consisting of O and NH; and Y is selected from the group consisting of O and NH;
provided that when X is O, then Y is O;
R¹ is selected from the group consisting of ethyl, ethoxycarbonylmethyl, 3-hydroxyphenyl, 4-hydroxyphenyl, 2-fluorophenyl, 2-chlorophenyl, 3-chlorophenyl, 3-fluorophenyl, 2-methylphenyl, 4-methylphenyl, 2-isopropylphenyl, 3-methoxyphenyl, 4-methoxyphenyl, 2-trifluoromethylphenyl, 3-trifluoromethylphenyl, 4-trifluoromethylphenyl, 3-nitrophenyl, 3-cyanophenyl, 4-cyanophenyl, 3-ethoxycarbonylphenyl, 4-hydroxy-2-methylphenyl, 3-chloro-4-methylphenyl, 4-methoxy-2-methylphenyl, 2,3-dimethylphenyl, 2,5-dimethylphenyl, 3,4-dimethylphenyl, 3,5-dimethylphenyl, 2,4-dimethoxphenyl, 2,5-dimethoxyphenyl, 2,4-dihydroxyphenyl, 3,4-dichlorophenyl, 2,5-dichlorophenyl, 2,4-difluorophenyl, 3,5-bis(trifluoromethyl)phenyl, 3,5-di(methoxycarbonyl)phenyl, 4-methylthiophenyl, 4-methylsulfonylphenyl and 1-naphthyl;
or a pharmaceutically acceptable salt thereof.

5. A pharmaceutical composition comprising a pharmaceutically acceptable carrier and a compound of claim 1.

6. A pharmaceutical composition made by mixing a compound of claim 1 and a pharmaceutically acceptable carrier.

7. A process for making a pharmaceutical composition comprising mixing a compound of claim 1 and a pharmaceutically acceptable carrier.

8. A method of treating a central nervous system a disorder comprising administering to a subject in need thereof a therapeutically effective amount of the compound of claim 1.

9. The method of claim 8, wherein the central nervous system disorder is selected from the group consisting of Alzheimer's Disease (AD), mild cognitive impairment, senility, dementia, dementia with Lewy bodies, Down's syndrome, dementia associated with Parkinson's disease and dementia associated with beta-amyloid.

10. The method of claim 8, wherein the central nervous system disorder is selected from Alzheimer's Disease or mild cognitive impairment.

11. The use of a compound as in claim 1 for the preparation of a medicament for treating: (a) Alzheimer's Disease (AD), (b) mild cognitive impairment, (c) senility, (d) dementia, (e) dementia with Lewy bodies, (f) Down's syndrome, (g) dementia associated with Parkinson's disease and (h) dementia associated with beta-amyloid, in a subject in need thereof.
